(19) United States
(12) Patent Application Publication
(10) Pub. No.: US 2018/0125730 A1
(43) Pub. Date: May 10, 2018

(54) Wicking and absorbent permeable liner with fluid release method

(71) Applicant: Flechelle Morin, Cardiff by the Sea, CA (US)

(72) Inventor: Flechelle Morin, Cardiff by the Sea, CA (US)

(21) Appl. No.: 15/862,019

(22) Filed: Jan. 4, 2018

(51) Int. Cl. A61F 13/537; A61F 13/15

(52) U.S. Cl. CPC A61F 13/537; A61F 13/539; A61F 13/15203

(57) ABSTRACT

A wicking and absorbent liner (liner) and a method for wicking, absorbing, and releasing menstrual flow and/or other bodily fluids is provided by this invention. The liner has fluid permeable body-facing and garment-facing surfaces, and one or more fluid absorbent core layers. The liner is designed to use in conjunction with sanitary napkins, cloth menstrual pads, panty liners, menstrual panties, and/or other absorbent feminine hygiene items (absorbent feminine hygiene items). The liner is designed and configured to fit between the genital area of a woman and the body facing surface of an absorbent feminine hygiene item.

### BACKGROUND OF THE INVENTION

Absorbent feminine hygiene items such as sanitary napkins, cloth menstrual pads, panty liners, and menstrual panties are designed to absorb and retain menstrual flow and other bodily fluids and discharges from a woman's genital area with the help of an impermeable backing to prevent leaks from soiling the wearer's clothing. Absorbent feminine hygiene items are worn by women on top of the crotch area of undergarments, held in place with some type of adhesive or fasteners. Absorbent feminine hygiene items come in a multitude of shapes, thickness and absorbability attributes. Absorbent feminine hygiene items such as menstrual panties are worn directly on the body, the same way a regular panty would be worn, and retain the menstrual flow within its absorbent core and impermeable outer layer.

In the past, a number of efforts have been directed at providing absorbent feminine hygiene items that would allow the bodily fluids to be absorbed away from the body. For example, in U.S. Pat. No. 7,307,197 issued to Mizutani, et al. on Dec. 11, 2007, and U.S. Pat. No. 7,329,243 issued to Mizutani, et al. on Feb. 12, 2008, the absorbent articles are inserted into and between the wearer's labia to absorb the bodily fluids before it has the chance to soil the wearer's garment. However, the possible dehydration to the labia that may be caused by an absorbent article being inserted into the gentle and sensitive labia area of a woman may not be an adequate choice for all women. The present invention is not an ‘interlabial pad’, but instead is a liner designed to be placed outside the wearer's labia area.

In another instance, U.S. Pat. No. 9,024,108 issued to McManus on May 5, 2015, has a “multi-element inner and outer pad apparatus and method”, and it states: “The inner disposable pad is constructed so as to not allow any menstrual liquid to come in contact with any part of the outer reusable pad”, and also states: “using an inner pad outer layer made of a breathable liquid barrier. Said breathable liquid barrier being capable of stopping and retaining all menstrual liquids . . . ”. Unlike U.S. Pat. No. 9,024,108, the present invention comprise of a single liner capable of wicking, absorbing and releasing menstrual flow and bodily fluids into any absorbent feminine hygiene items. Additionally, U.S. Pat. No. 9,024,108 in claim 3 states that the user can use “a supplemental disposable inner pad being placed between said inner pad and the user's exterior vaginal surfaces and said supplemental disposable inner pad having the same materials as said inner pad.” Furthermore, in the Summary of Invention of U.S. Pat. No. 9,024,108, it states that: “Optionally, at the discretion of the user, the user may purchase and place between her vaginal surfaces and the aforesaid regular inner pad, an additional supplemental inner pad, comprised of the same uniquely combined viscoelastic hydrophilic materials, but not having a liquid barrier back surface . . . ”. Although the user can purchase a permeable supplemental inner pad, this said supplemental inner pad is designed to be used and worn exclusively with the regular inner pad and outer pad of the same invention. The invention presented here allows a woman to use this liner with any disposable or washable absorbent feminine hygiene items she prefers, making it easier to fit the many different shapes and sizes of different women, for both comfort and effectiveness.

Other examples of inventions made to absorb and wick the bodily fluids into an absorbing pad are U.S. Pat. No. 7,530,973 issued to Tanio, et al. on May 12, 2009, and U.S. Pat. No. 5,624,421 issued to Dabi, et al. on Apr. 29, 1997, which respectively features three-dimensional structures and flexible hydrophilic wick means integrated on the skin-side surface of a pad/napkin. Unlike both these inventions which respectively integrates the absorbent three-dimensional structures and the flexible hydrophilic wick means into a sanitary napkin, the present invention is not part of a sanitary napkin, but is designed as a separate wicking and absorbent liner that wicks, absorb and releases the menstrual flow and fluids through its permeable body-facing surface, its one or more fluid absorbent core layers, and through its liquid permeable garment-facing surface into any woman's disposable or washable absorbent feminine hygiene items of her choice, therefore allowing a woman the opportunity to select the best absorbent feminine hygiene items to satisfy her specific needs, which on varied days may be a thin sanitary napkin, or an elongated thick overnight pad, or a menstrual panty, et cetera.

In U.S. Pat. No. 6,582,411 issued to Carstens, et al. on Jun. 24, 2003, says to be a “highly efficient absorbent article for use with menstrual pant”, also states that it has a “liquid resistant, or liquid impermeable backsheet joined to the topsheet”. Unlike U.S. Pat. No. 6,582,411, the present invention has a liquid permeable garment-facing surface, from which the menstrual flow and bodily fluids are freely allowed to be released onto, and be absorbed by the wearer's absorbent feminine hygiene items of her choice.

The feminine hygiene products industry has lacked to offer women a viable option to wick, absorb, and release their menstrual flow in a fashion that permits them to protect their clothing, as well as to prolong the lifecycle of their absorbent feminine hygiene items. The liner in this invention is also friendly to the environment in the fact that it limits the numbers of absorbent feminine hygiene items with impermeable lining to reach the landfills.

Consequently, there is a need in the art to have a wicking and absorbent liner that will wick, absorb and release the menstrual flow and fluids into absorbent feminine hygiene items.

### DESCRIPTION

### References Sited

| | | |
|---|---|---|
| 7,307,197 | Dec. 11, 2007 | Mizutani, et al. |
| 7,329,243 | Feb. 12, 2008 | Mizutani, et al. |
| 9,024,108 | May 5, 2015 | MacManus |
| 7,530,973 | May 12, 2009 | Tanio, et al. |
| 5,624,421 | Apr. 29, 1997 | Dabi, et al. |
| 6,582,411 | Jun. 24, 2003 | Carstens, et al. |

### FIELD OF THE INVENTION

The present invention relates to absorbent feminine hygiene items such as sanitary napkins, panty liners, cloth menstrual pads, and other absorbent garments such as menstrual panties, and the like. The present invention relates to a liner capable of wicking, absorbing and releasing the menstrual flow and bodily fluids into absorbent feminine hygiene items without restrictions.

### BRIEF SUMMARY OF THE INVENTION

The liner in this invention is made to wick the menstrual flow and bodily fluids away from a woman's body through its non-restrictive permeable body-facing surface, absorb the said flow and fluids within its one or more fluid absorbent core layers, and direct the said flow and fluids through its non-restrictive permeable garment-facing surface into an absorbent feminine hygiene item. The liner in this invention is made to be worn in conjunction with other absorbent feminine hygiene items. The uniqueness of this said liner is in its permeable garment-facing surface which allows the flow and fluids to freely be absorbed into an absorbent feminine hygiene item. Furthermore, additional advantages to the wearer include, but not limited to, the increase lifecycle of the absorbent feminine hygiene items used in conjunction with the liner in this invention, and the cost-saving associated with the reduced use of absorbent feminine hygiene items as the woman can replace the liner in this invention many times before having to replace their absorbent feminine hygiene items.

### DESCRIPTION OF THE DRAWINGS

In the drawings:

FIG. 1 is a plan view of a preferred embodiment of the invention. There are two drawings in FIG. 1; FIG. 1A, and FIG. 1B. The drawing in FIG. 1A as viewed depicts the preferred embodiment of the body-facing surface of the liner, the core fluid absorbent layer(s), and the garment-facing inner-surface of the liner. The drawing in FIG. 1B as viewed depicts the preferred embodiment of the garment-facing surface of the liner. The dashed lines depict one of many options for the disposition of the adhesive elements.

FIG. 2 is a plan view of the side of the liner. The dashed lines depict additional various thicknesses of the fluid absorbent core layers of the liner.

### DETAILED DESCRIPTION OF THE DRAWINGS

For the purpose of promoting an understanding of the principles of the invention, reference will now be made to the embodiments illustrated in the drawings. These embodiments descriptions are for the purpose of describing, but not limiting the invention. It will be understood that no limitation of the scope of the invention is thereby intended, such alterations and further modifications in the illustrated device, and such further applications of the principles of the invention as illustrated herein being contemplated as would normally occur to one skilled in the art to which the invention relates.

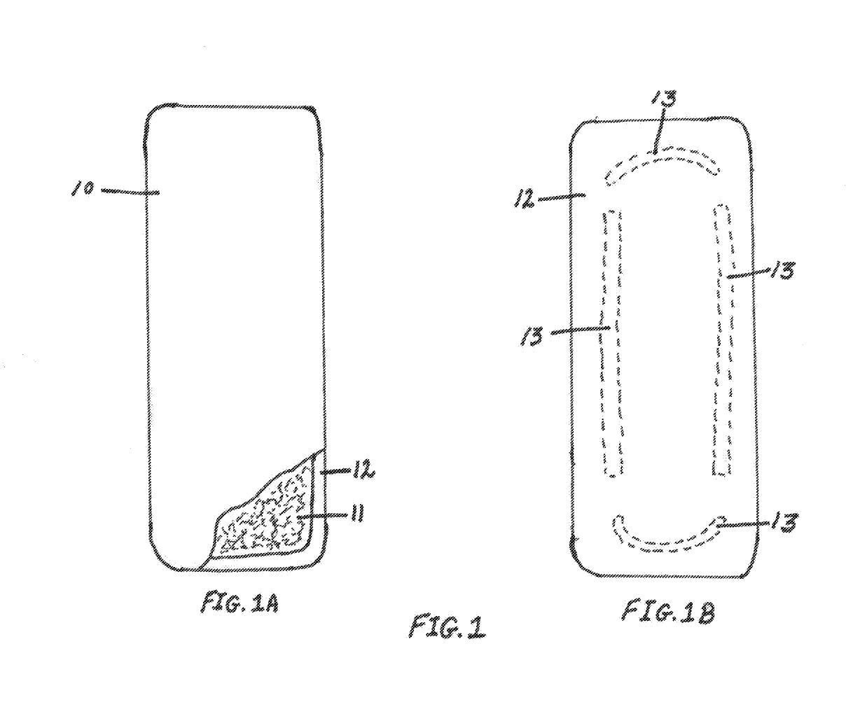

With reference to FIG. 1, 1A, the liner of the present invention comprises a body 10 having a fluid permeable body-facing surface, a fluid absorbent core layer 11, disposed between the body-facing surface and the garment-facing surface 10, 12, respectively. FIG. 1, 1B, the liner of the present invention comprises a body 12 having a fluid permeable garment-facing surface, and 13 preferred adhesive zones.

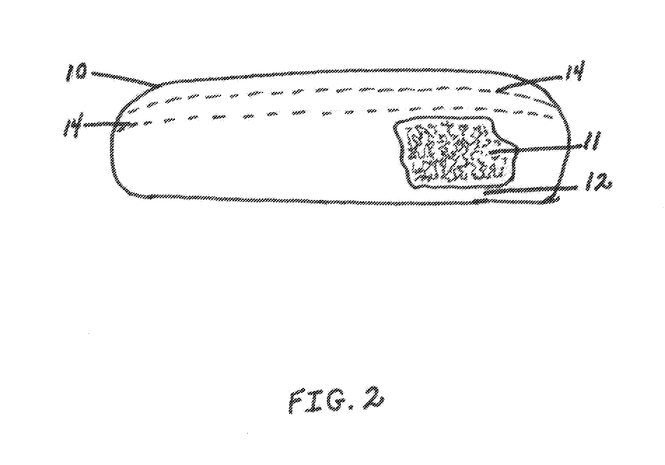

With reference to FIG. 2, the liner of the present invention comprises a body 10 having a fluid permeable body-facing surface, 12 having a fluid permeable garment-facing surface, 11 having a fluid absorbent core layer disposed between the body-facing surface and the garment-facing surface 10, 12, respectively. The liner of the present invention may include possible additional fluid absorbent core layers to offer additional absorbency, presented here with dotted lines 14.

### DETAILED DESCRIPTION OF THE INVENTION

The present invention relates to a wicking and absorbent permeable liner with fluid release method made for wearing in conjunction with other absorbent feminine hygiene items.

This present invention is of a wicking and absorbent liner comprising a body-facing side, and garment-facing side, and one or more fluid absorbent core layers. The body-facing and garment-facing surface are permeable to menstrual flow and fluids. The body-facing surface is meant to wick away the flow and fluids from the woman's body. The one or more fluid absorbent core layers are meant to absorb and hold the flow and fluids before it is released through the permeable garment-facing surface. The permeable garment-facing surface is meant to allow the flow and fluids to be released freely into an absorbent feminine hygiene item. The liner may comprise of organic or non-organic disposable materials, or made of organic or non-organic cloth or other washable materials. The absorbent liner in this present invention is not limited to one thickness, but may comprise of one or more fluid absorbent core layers to produce absorbent liners with varying thicknesses and absorbability as to satisfy the need to absorb a light, medium, or heavy menstrual flow, or other bodily fluids.

The liner in this invention is designed and configured to fit between the genital area of a woman and the body-facing part of an absorbent feminine hygiene item. This liner is meant to fill the gap often found between an absorbent feminine hygiene item and a woman's body, and is meant to substantially maintain contact with the labial and genital area of a woman, and in doing so, is meant to improve the absorption rate by wicking the menstrual flow and fluids, improve the collection and absorption of said flow and fluids, and improve the direction of said flow and fluids by releasing the said flow and fluids to the wanted end-point which is an absorbent feminine hygiene item. The said liner is meant to be worn on top of the longitudinal body-facing part of an absorbent feminine hygiene item, and be worn on top of the crotch area of a menstrual panty.

The body-facing side is meant to be worn body facing. In one embodiment, the body-facing surface may be made of soft, non-irritant fluid permeable material, and may or may not be attached to the one or more fluid absorbent core layers. In another embodiment, the body-facing surface may be an extension of the upper-facing fluid absorbent core layer, forming one unit with the one or more fluid absorbent layers. In another embodiment, the body-facing surface may be attached to the garment-facing surface, forming one surrounding unit around one or more fluid absorbent core layers. The body-facing surface is meant to gently press on the outer labia, without penetrating the inner labia as to not dehydrate and irritate the sensitive inner labia. In some embodiments, because of the pliability and softness of the said absorbent liner, the absorbent liner is capable to comfortably adapt to the configuration of the woman's genital area. These above-mentioned embodiments are just some of the possible versions of this invention and should not be construed as being limited to.

The garment-facing side is meant to be worn garment facing. In one embodiment, the garment-facing surface may be made of soft, non-irritant permeable material, and may or may not be attached to the one or more fluid absorbent core layers. In another embodiment, the garment-facing surface may be an extension of the lower-facing fluid absorbent layer, forming one unit with the one or more fluid absorbent layers. In another embodiment, the garment-facing surface may be attached to the body-facing surface, forming one surrounding unit around one or more fluid absorbent core layers. The garment-facing surface is meant to be worn on top of an absorbent feminine hygiene item, or worn on top of the crotch area of a menstrual panty. In some embodiments, because of the pliability and softness of the said absorbent liner, the absorbent liner is capable to adapt to the configuration of the absorbent feminine hygiene item. These above-mentioned embodiments are just some of the possible versions of this invention and should not be construed as being limited to.

In one embodiment, the garment-side of the absorbent liner includes one or more adhesive material strips, dots, circles or semi-circles. The adhesive element added to the garment-facing surface is situated as to not interfere, or if so minimally, with the release of the flow and fluids into an absorbent feminine hygiene item. In another embodiment, the method of attachment of the absorbent liner to an absorbent feminine hygiene item can comprise, but not limited to ties, loops, snaps, buttons, fabric hook and loop fasteners.

In the said embodiment where the garment-side of the absorbent liner includes one or more strips, dots, circles or semi-circles of adhesive material, these said adhesive elements are covered by one or more protective strips, such as but not limited to, what is known to the art as release strips. These release strips, which may be in the form of a strip, a dot, circle or a semi-circle are added to cover the adhesive element(s) to protect them before use. The adhesive strips are put into place as to ensure that no adhesive enters in contact with the woman's body.

The adhesive element, or elements, may be situated either at the center of the garment-facing side of the liner, or at the sides of the garment-facing side of the liner, or at each ends of the garment-facing side of the liner, or a combination of these above-mentioned placements.

The adhesive element, or elements, of the liner is put into place as to attach the liner to the body-facing part of absorbent feminine hygiene items, or on the crutch area of absorbent menstrual panties.

Because most absorbent feminine hygiene items are intended to be worn within a pair of panties and/or undergarments, they are made relatively small and particularly narrow to accommodate the fit, and due to their inherent small size, they make it hazardous for the menstrual flow and bodily fluids to adequately reach the surface of these absorbent feminine hygiene items. Furthermore, the woman may misjudge the placement of such a sanitary napkin, and in so doing compromise the satisfactory collection and absorption of the menstrual flow and bodily fluids, allowing leaks and leaving the woman with soiled garments.

The liner in this present invention prevents unwanted menstrual flow and fluids leaks caused by the inherent narrowness of most absorbent feminine hygiene items, especially during a heavy menstrual flow, or a sudden release of flow when a woman gets up after having sat for a while, or having laid down. The said liner wicks the menstrual flow and bodily fluids through its body-facing permeable surface as soon as the said flow reaches the outside labial area of a woman; then the said liner absorbs the wicked menstrual flow and fluids within its one or more fluid absorbent core layers; then the said absorbent liner leads and releases the flow and fluids through its garment-facing fluid permeable surface to the center surface of an absorbent feminine hygiene item, reducing the risk of the menstrual flow to leak to the side, or sides, of the absorbent feminine hygiene item. Furthermore, the said liner in this invention absorbs the menstrual flow into its one or more fluid absorbent core layers before the said flow and fluids is released to the absorbent feminine hygiene items through its garment-facing fluid permeable surface, adding to the lifecycle of the absorbent feminine hygiene items; the woman can replace the said liner many times before having to replace the absorbent feminine hygiene item she is wearing.

Not only does this present invention helps protect women against having their menstrual flow and bodily fluids to leak from their absorbent feminine hygiene items of choice into their clothing, it also allows women to use their favorite absorbent feminine hygiene items, no matter the abundance of their flow. This invention allows women to no longer be limited to wear super tick, long and absorbent feminine hygiene items during their heavy menstrual flow days; they can use a thinner, slimmer, less invasive absorbent feminine hygiene item, therefore can continue to wear their tight-fitting clothing even during their heaviest menstrual flow days. This invention offers women a unique versatility in the absorbent feminine hygiene items they wear, in an industry that otherwise limits women to use a specifically designed absorbent menstrual pad dependent on their menstrual flow.

I claim:

1. A wicking and absorbent liner comprising a fluid permeable body-facing surface, one or more fluid absorbent core layers, and a fluid permeable garment-facing surface;
said liner is capable to wick the menstrual flow and bodily fluids away from the labial and genital area of a woman with its fluid permeable body-facing surface;
said liner comprising of one or more fluid absorbent core layers;
said liner is capable to absorb the menstrual flow and bodily fluids into its one or more fluid absorbent core layers;
said liner is designed to release the said flow and fluids through its fluid permeable garment-facing side;
said liner meant to be worn on top of the longitudinal body-facing part of absorbent feminine hygiene items, and/or the crotch area of menstrual panties, or other absorbent menstrual items of the like;
said liner to touch and cover part of the surface of an absorbent feminine hygiene item, and/or part of the crotch area of a menstrual panty, or other absorbent menstrual items of the like;
said liner to touch and cover at least a portion of the exterior surface of the labial and genital area of a woman;
said liner can be worn with other menstrual feminine hygiene items;
said liner to substantially maintain contact with the labial and genital area of a woman;
said liner is capable to adapt to the configuration of the woman's genital area;
said liner is capable to adapt to the configuration of absorbent feminine hygiene items;
said liner is designed to release the menstrual flow and bodily fluids into the longitudinal body-facing part of absorbent feminine hygiene items.

2. The liner of claim 1, wherein said liner comprise of a fluid permeable body-facing surface, a fluid permeable garment-facing surface, and one or more fluid absorbent core layers, said surfaces and fluid absorbent core layers are effective at wicking, absorbing, and releasing menstrual flow and bodily fluids.

3. The liner of claim 1, further comprising of a method of construction using one or more fluid absorbent core layers to produce additional absorbency with varying thicknesses as to satisfy the need to absorb a light, medium, or heavy menstrual flow, or other bodily fluids.

4. A liner comprising one or more pressure-sensitive adhesive element, or elements, placed on the garment-facing side of the said liner, herein the pressure-sensitive adhesive element, or elements, use for affixing the liner to the longitudinal body-facing part of an absorbent feminine hygiene item, and said pressure-sensitive adhesive element, or elements, is positioned in such a way to allow the menstrual flow or bodily fluids to go through its permeable garment-facing side and enter in contact with the wearer's absorbent feminine hygiene item.

5. The liner of claim 4, wherein said liner comprise of an adhesive protecting paper release covering the adhesive element, or elements, to protect the adhesive before use.
